(19) United States
(12) Patent Application Publication
(10) Pub. No.: US 2015/0342776 A1
(43) Pub. Date: Dec. 3, 2015

(54) Adjustable Supporter

(71) Applicant: Brigit ANDERSON, Scottsdale, AZ (US)

(72) Inventor: Brigit ANDERSON, Scottsdale, AZ (US)

(21) Appl. No.: 14/290,918

(22) Filed: May 29, 2014

(51) Int. Cl. A61F 5/40

(52) U.S. Cl. CPC A61F 5/40

(57) ABSTRACT

Aspects of the present invention pertain to men's apparel, and specifically, men's undergarments. Aspects of the present invention pertain to an adjustable supporter.

### FIELD OF THE INVENTION

Embodiments of the present invention generally relate to men's apparel, and specifically, men's undergarments.

### BACKGROUND

Testicular trauma is defined as any injury sustained by the testicle. Types of injuries include blunt, penetrating, or degloving. Blunt trauma refers to injuries sustained from objects applied with any significant force to the scrotum, testicles, or pelvic region. This can occur with various types of activity. Examples include a kick to the groin or an injury caused by a sports ball, such as a baseball, golf ball, football, or the like. Penetrating trauma refers to injuries sustained from sharp objects or high-velocity missiles. Examples include gunshot and stab wounds. Degloving injuries (or avulsion injuries) are less common and involve situations where scrotal skin is sheared off, for example, when a testicle becomes trapped in heavy machinery.

Testicular rupture or fractured testis refers to a rip or tear in the tunica albuginea resulting in extrusion of the testicular contents. Testicular dislocation is an uncommon and sometimes easily overlooked event that refers to a testis that has been relocated from its orthotopic position to another location secondary to blunt trauma. Most cases of testicular dislocation are the result of motorcycle crashes, and one third involve both testicles. This is related to impact with the fuel tank, and the inguinal region is the most frequent site of displacement.

The most common cause of blunt testicular trauma is sports injuries. The second most common cause of testicular trauma is a kick to the groin. Less common etiologies include motor vehicle accidents, falls, and straddle injuries. Pelvic injury can cause significant testicular/scrotal edema. The most common cause of penetrating testicular injuries is a gunshot wound to the genital area. Other causes include stab wounds, self-mutilation, animal bites (usually dog), and emasculation. The most common cause of degloving testicular injuries is accidents incurred while operating heavy machinery (i.e., industrial or farming accidents).

Men that have undergone successful medical treatment after enduring testicular trauma may be advised by a urologist to wear an athletic supporter that will provide additional support for the genitalia either while undergoing therapy or while engaging in physical activities. An athletic supporter (also known as a jock, strap, supporter, or jock strap) is an undergarment designed for supporting the male genitalia during sports or other vigorous physical activity.

However, because of the various forms and degrees of testicular, scrotal, or pelvic trauma, a typical athletic supporter may not provide the necessary comfort and support needed for the genitalia of a trauma victim. For example, for comfort, it may be necessary to elevate or decline the injured testicle or scrotum due to pelvic trauma or increased edema in the scrotal/testicular region. Athletic supporters do not address that problem, leaving the trauma victim to either endure the healing process in pain, reduce physical activity, engage in athletic activities without adequate protection or in discomfort, or not to engage in any activity at all.

### SUMMARY

Aspects of the present invention include a supporter comprising a waistband, a support pouch secured to the waistband, and at least one strap secured to the support pouch, whereby the opposite ends of the strap are adjustably secured to the waistband.

Aspects of the present invention include a supporter comprising a waistband, a support pouch secured to the waistband, a first strap secured to the support pouch, whereby the opposite end of the first strap is adjustably secured to the waistband, and a second strap secured to the support pouch, whereby the opposite end of the second strap is adjustably secured to the waistband.

Aspects of the present invention include a method of treating male genital trauma comprising the steps of placing male genitalia in a support pouch secured to a waistband, and adjusting the position of the support pouch by adjustably securing opposite ends of at least one strap secured to the base of the support pouch to the waistband.

### BRIEF DESCRIPTION OF THE DRAWINGS

Although the scope of the present invention is much broader than any particular embodiment, a detailed description of the preferred embodiment follows together with drawings. These drawings are for illustration purposes only and are not drawn to scale. Like numbers represent like features and components in the drawings. The invention may best be understood by reference to the ensuing detailed description in conjunction with the drawings in which:

FIG. 1 illustrates an exemplary adjustable athletic supporter.

FIG. 2 illustrates an exemplary adjustable athletic supporter.

FIG. 3 illustrates an exemplary adjustable athletic supporter.

FIG. 4 depicts a magnified left frontal view of an exemplary embodiment of the present invention.

### DETAILED DESCRIPTION

The embodiments of the present invention are described more fully hereinafter with reference to the accompanying drawings, which form a part hereof, and which show, by way of illustration, specific exemplary embodiments by which the invention may be practiced. This invention may, however, be embodied in many different forms and should not be construed as limited to the embodiments set forth herein. Rather, the disclosed embodiments are provided so that this disclosure will be thorough and complete, and will fully convey the scope of the invention to those skilled in the art.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

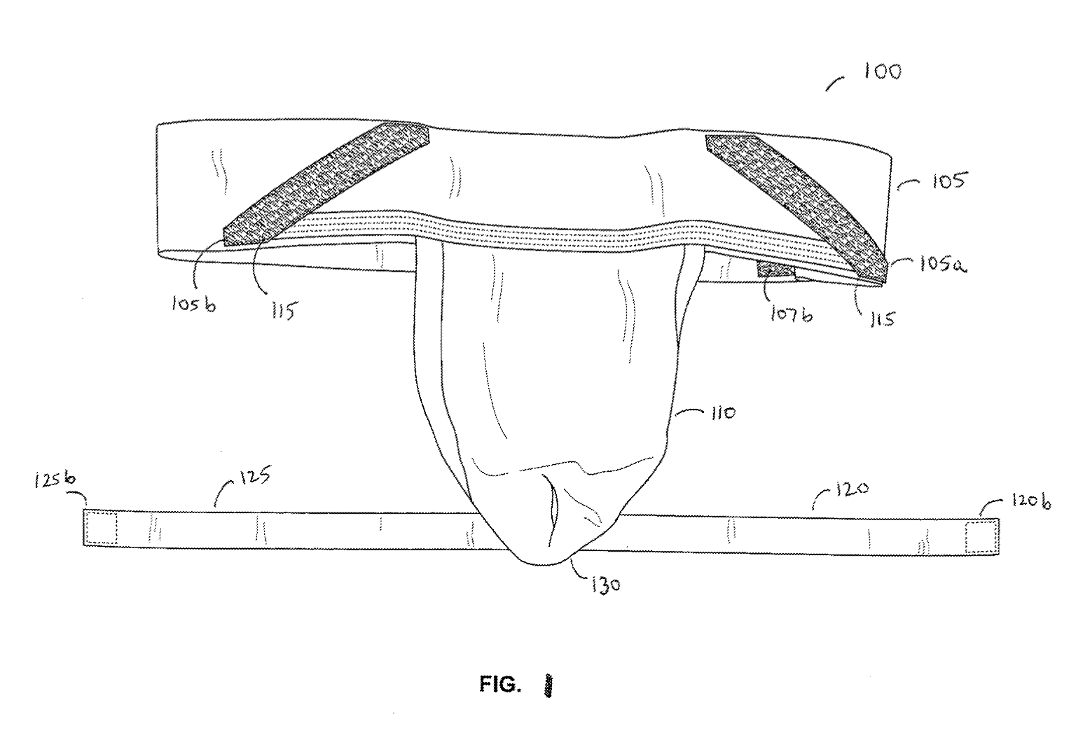

FIG. 1 depicts an exemplary embodiment of the present invention. In one embodiment, athletic supporter 100 includes a waistband 105. Waistband 105 may be made of any material, such as, for example, cotton, spandex, etc. In one embodiment, waistband 105 is made of elastic. In another embodiment, waistband 105 may be stitched in one-piece, or may be assembled from multiple pieces. In another embodiment, waistband 105 may be adjustably secured by hook-and-loop fasteners 107a (not shown) and 107b such as Velcro, or any other adjustable means, to allow for additional comfort to accommodate to the expanding or contracting waistlines of the user. Support pouch 110 adapted to house the user's genitalia is secured to the front of waistband 105. Support pouch 110 may be made of any suitable material and may be designed in any shape or size. In one embodiment, support pouch 110 is formed as an envelope with an aperture (not shown) to receive an optional cup to further protect the genitalia. In one embodiment, support pouch 110 may be detachably and adjustably secured to waistband 105, such as, for example, by way of hook-and-loop fasteners such as Velcro, or any other adjustably secured form. Straps 120 and 125 may be secured to the base 130 of support pouch 110. In one embodiment, the opposite ends 120b and 125b of straps 120 and 125 may be detachably and adjustably secured to left 105a and right 105b sides of waistband 105, by for example, hook-and-loop fasteners 115 such as Velcro, or any other adjustably secured form, such as buttons. In another embodiment, a single strap is used and is secured to the base of support pouch 130, at, for example, the strap's midpoint. In another embodiment, suspenders may be detachably and adjustably secured to waistband 105.

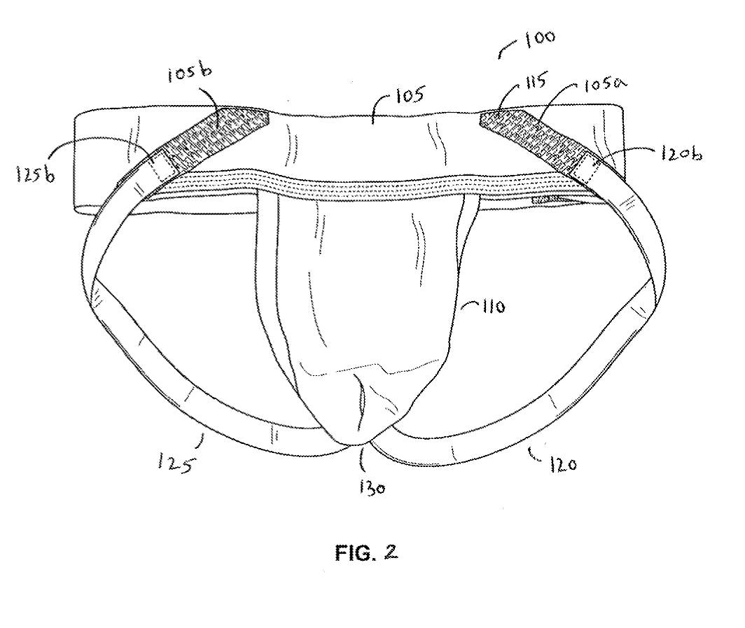

FIG. 2 depicts an exemplary embodiment of the present invention where the ends of straps 120 and 125 are detachably and adjustably secured to the left 105a and right 105b sides of waistband 105, by for example, hook-and-loop fasteners 115 such as Velcro, or any other adjustably secured form.

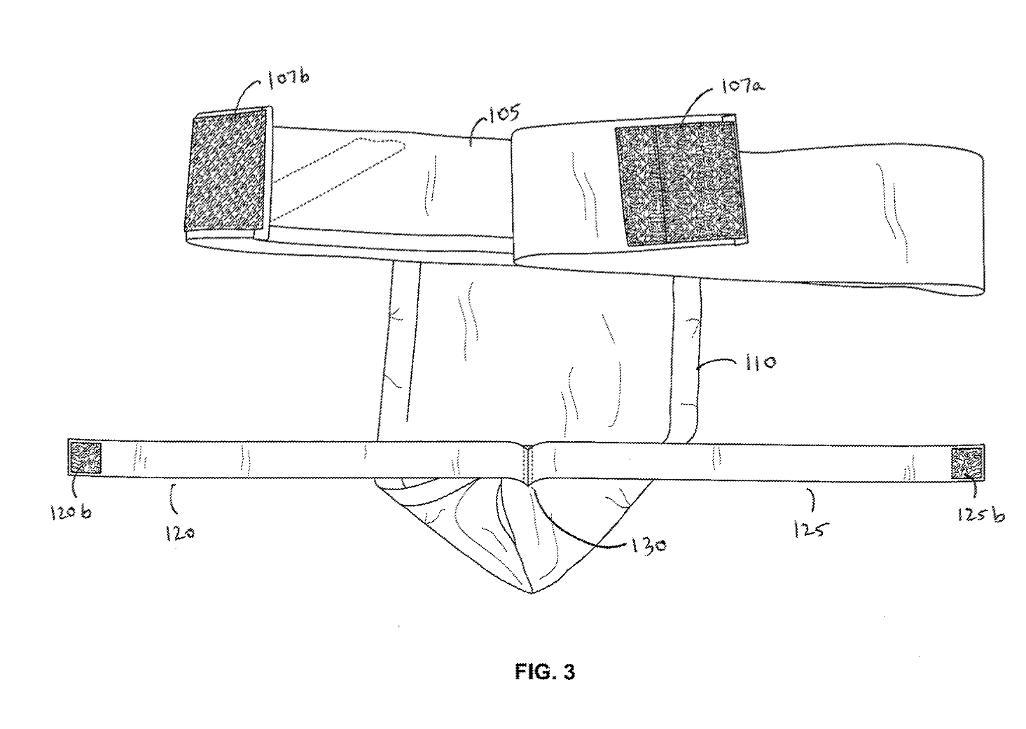

FIG. 3 depicts an exemplary embodiment of the present invention. In one embodiment, waistband 105 may be stitched in one-piece, or may be assembled from multiple pieces. In another embodiment, waistband 105 may be adjustably secured by hook-and-loop fasteners 107a and 107b such as Velcro, or any other adjustable means, to allow for additional comfort to accommodate to the expanding or contracting waistlines of the user.

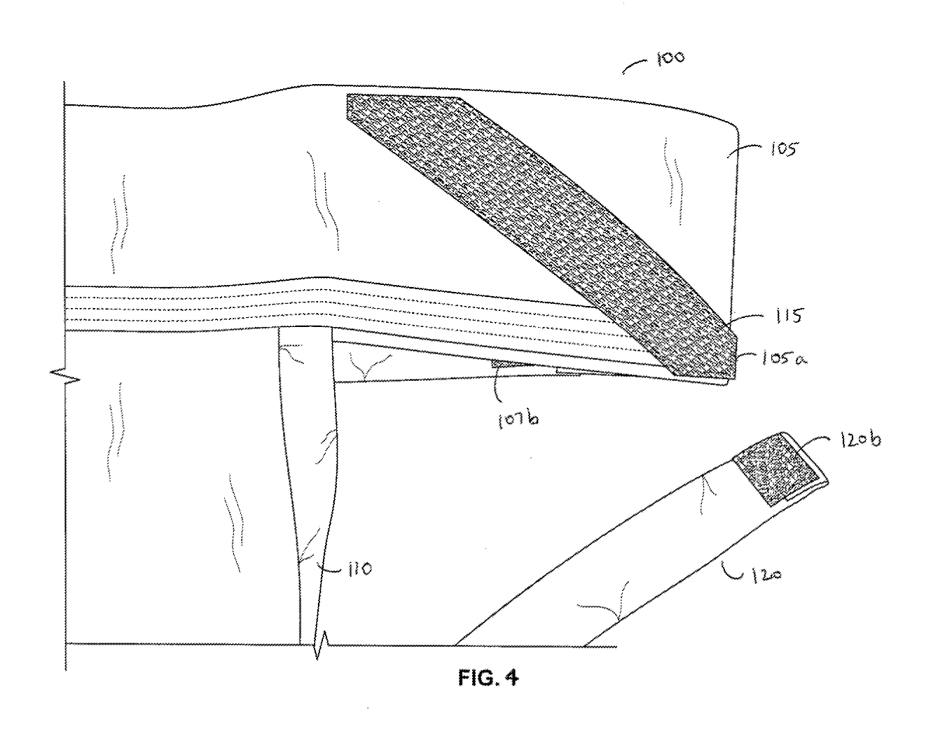

FIG. 4 depicts a magnified left frontal view of an exemplary embodiment of the present invention. In one embodiment, athletic supporter 100 includes a waistband 105. In one embodiment, waistband 105 may be adjustably secured by hook-and-loop fasteners 107a (not shown) and 107b such as Velcro, or any other adjustable means, to allow for additional comfort to accommodate to the expanding or contracting waistlines of the user. Support pouch 110 adapted to house the user's genitalia is secured to the front of waistband 105. In one embodiment, the opposite end 120b of strap 120 may be detachably and adjustably secured to the left 105a side of waistband 105, by for example, hook-and-loop fasteners 115 such as Velcro, or any other adjustably secured form, such as buttons.

Although a specific embodiment of the present invention has been described, it will be understood by those of skill in the art they are not intended to be exhaustive or to limit the invention to the precise forms disclosed and obviously many modifications and variations are possible in view of the above teachings, including equivalents. Accordingly, it is to be understood that the invention is not to be limited by the specific illustrated embodiments, but only by the scope of the appended claims.

I claim:

1. A supporter comprising:
a waistband;
a support pouch secured to the waistband; and
at least one strap secured to the support pouch, whereby the opposite ends of the strap are adjustably secured to the waistband.

2. The supporter of claim 1, wherein the waistband is adjustably secured.

3. The supporter of claim 1, wherein the support pouch is adjustably secured to the waistband.

4. The supporter of claim 1, wherein the strap is secured to the base of the support pouch.

5. The supporter of claim 1, wherein two straps are secured to the base of the support pouch.

6. The supporter of claim 1, wherein the support pouch is adapted to receive a cup.

7. A supporter comprising:
a waistband;
a support pouch secured to the waistband;
a first strap secured to the support pouch, whereby the opposite end of the first strap is adjustably secured to the waistband; and
a second strap secured to the support pouch, whereby the opposite end of the second strap is adjustably secured to the waistband.

8. The supporter of claim 7, wherein the waistband is adjustably secured.

9. The supporter of claim 7, wherein the support pouch is adjustably secured to the waistband.

10. The supporter of claim 7, wherein the first strap is secured to the base of the support pouch.

11. The supporter of claim 7, wherein the second strap is secured to the base of the support pouch.

12. The supporter of claim 7, wherein the support pouch is adapted to receive a cup.

13. A method of treating male genital trauma comprising the steps of:
placing male genitalia in a support pouch secured to a waistband; and
adjusting the position of the support pouch by adjustably securing opposite ends of at east one strap secured to the base of the support pouch to the waistband.

14. The method of claim 13, wherein the position of the support pouch is adjusted by raising the left side of the support pouch by adjustably securing the left strap at a higher position on the waistband.

15. The method of claim 13, wherein the position of the support pouch is adjusted by raising the right side of the support pouch by adjustably securing the right strap at a higher position on the waistband.

16. The method of claim 13, wherein the position of the support pouch is adjusted by lowering the left side of the support pouch by adjustably securing the left strap at a lower position on the waistband.

17. The method of claim 13, wherein the position of the support pouch is adjusted by lowering the right side of the support pouch by adjustably securing the right strap at a lower position on the waistband.

18. The method of claim 13, wherein the position of the support pouch is adjusted by raising the support pouch by adjustably securing the left and right straps at higher positions on the waistband.

19. The method of claim 13, wherein the position of the support pouch is adjusted by lowering the support pouch by adjustably securing the left and right straps at lower positions on the waistband.
